(19) United States
(12) Patent Application Publication
(10) Pub. No.: US 2011/0077512 A1
(43) Pub. Date: Mar. 31, 2011

(54) Biopsy marker composition and method of use

(75) Inventor: John S. Boswell, Fresno, CA (US)

(73) Assignee: Dept. of Veterans Affairs

(21) Appl. No.: 12/801,605

(22) Filed: Jun. 16, 2010

Related U.S. Application Data
(60) Provisional application No. 61/187,446, filed on Jun. 16, 2009.

(51) Int. Cl. A61B 6/00; A61K 49/00

(52) U.S. Cl. 600/431; 600/476; 424/9.6

(57) ABSTRACT

An aqueous suspension of titanium dioxide, polymethylmethacrylate and Vitamin E oil is used to mark a location wherein a skin biopsy has been taken to enable subsequent identification of the biopsy location under ultraviolet light. The location is advantageously treated with Vitamin E oil on a daily basis subsequently extend the fluorescing life of the applied titanium dioxide.

### CROSS-REFERENCE TO RELATED APPLICATION

The present application derives from U.S. provisional application Ser. No. 61/187,446, filed Jun. 16, 2009, the priority of which is hereby claimed.

### BACKGROUND OF INVENTION

1. Field of Invention

The present invention relates to the field of medicine, and in particular (although not exclusively) to the specialized practices of dermatology and surgery where locating areas of interest on (or in) a human (or animal) need to be marked.

2. The Prior Art

In dermatology it is routine to perform shave biopsies at locations where skin cancer is suspected. The suspect location, typically about 1 centimeter in size, is usually injected with an anesthetic such as lidocaine with epinephrine, and then shaved off (see FIG. 1).

Directly following such a shave biopsy, bleeding of the wound may be stopped by application of a composition such as aluminum chloride to the wound bed with a cotton tip applicator, or by using electrocautery to “zap” the area and stop any bleeding. An antibiotic or other ointment is then applied, followed by a bandage. The patient is then discharged.

The actual biopsy specimen is usually then sent to another location for a pathology analysis, which results in a report. If the report confirms a diagnosis of skin cancer, the patient may return to the dermatologist, or may be referred to another dermatologist or plastic surgeon, for treatment. This treatment typically involves a wide excision of the skin cancer, with about 4 millimeters of normal skin included around the biopsy scar. The wide excised specimen is also submitted for pathology to ensure all the cancer has been removed.

It typically takes 1-2 weeks to get the initial biopsy report back, and then another few weeks, if not months, to subsequently schedule the patient for surgical excision. Thus, by the time the patient returns for the excision, the wound from the initial biopsy may have healed, making it difficult for the dermatologist or plastic surgeon to locate the exact location of the biopsy scar. The more time that passes between the initial biopsy and the excision, the more difficult it may be to determine the location of the initial biopsy. The problem of determining this location may be compounded for older patients who may have numerous scars, and/or if the anatomic location of the report is vague (e.g., “left arm”), and/or if the patient cannot recall exactly where the initial biopsy was taken. It is time consuming and laborious for the dermatologist to follow measurements and landmarks from a report, and impractical to excise every little scar he sees on the “left arm” or “back.”

Potential problems that may result from being unable to relocate the biopsy site include having to redo the biopsy (spending additional time and money, and delaying the treatment of cancer); substandard treatment (e.g., topical 5-fluorouracil or imiquimod instead of surgical excision of invasive SCC); treating the wrong site, etc.

One solution is to take photographs at the time of the initial biopsy. However, photographs taken during the biopsy process to identify the site are not always reliable. They may be lost or misplaced, they may not have good resolution (or may not clearly show the location of the biopsy), and/or they may not provide enough context relative to other tissues to identify the location of the biopsy.

It is known to mark a location of medical interest on a person's skin using an organic compound such as fluorescamine which will be invisible in ordinary light but will fluoresce under ultraviolet light. See, for example, U.S. Pat. Nos. 4,572,831 and 4,610,806. However, these compounds have been found to have a limited useful lifetimes as markers (a few weeks). An improved marker composition (and application technique) which will lengthen the time that the site of application will be visible under ultraviolet light is desired.

### SUMMARY OF THE INVENTION

I have discovered that an aqueous suspension of titanium dioxide and polymethylmethacrylate (PMMA) applied to the location to be marked, either by surface contact or injection beneath the surface, will provide a marker that will be invisible in ordinary light, but visible (fluoresce) under ultraviolet light, for an extended period of time. I have also discovered that when vitamin E oil is added to the aqueous suspension and/or applied to the spot on the skin immediately after the application of the T₁O₂-PMMA aqueous suspension, and preferably reapplied on a daily basis, the effectiveness of the T₁O₂-PMMA aqueous suspension will be extended to 12 or more weeks. The invention can be used to mark sites in a patient during surgery for subsequent location as necessary.

The invention will be more apparent from a review of the attached drawings, taken in conjunction with the following discussion.

### BRIEF DESCRIPTION OF THE DRAWINGS

FIG. 1 illustrates the shaving (removal) of a surface section of skin in a prior art technique,

FIG. 2 shows photographs of an untreated (control) biopsy location under normal and ultraviolet light at 1, 2, 3, 4, 8 and 12 week intervals,

FIG. 3 shows photographs of a biopsy location of a patient under normal and ultraviolet light at 1, 2, 3, 4, 8 and 12 week intervals when the biopsy location was treated with a titanium dioxide-PMMA suspension according to the present invention, and the location was thereafter treated with Vitamin E oil, and

FIG. 4 shows the application of the titanium dioxide-PMMA aqueous suspension to the skin of a person for marker purposes using a sterile tip applicator.

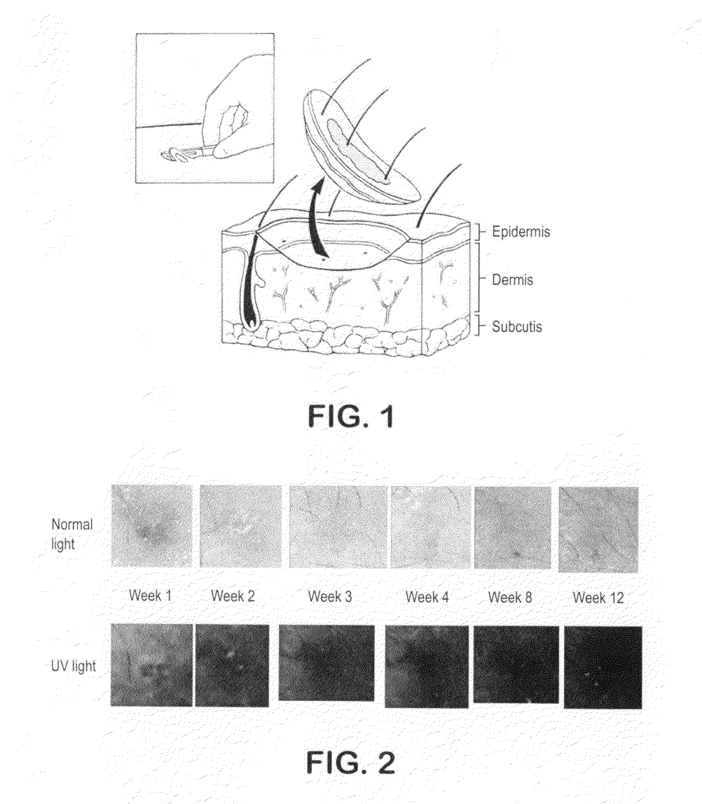

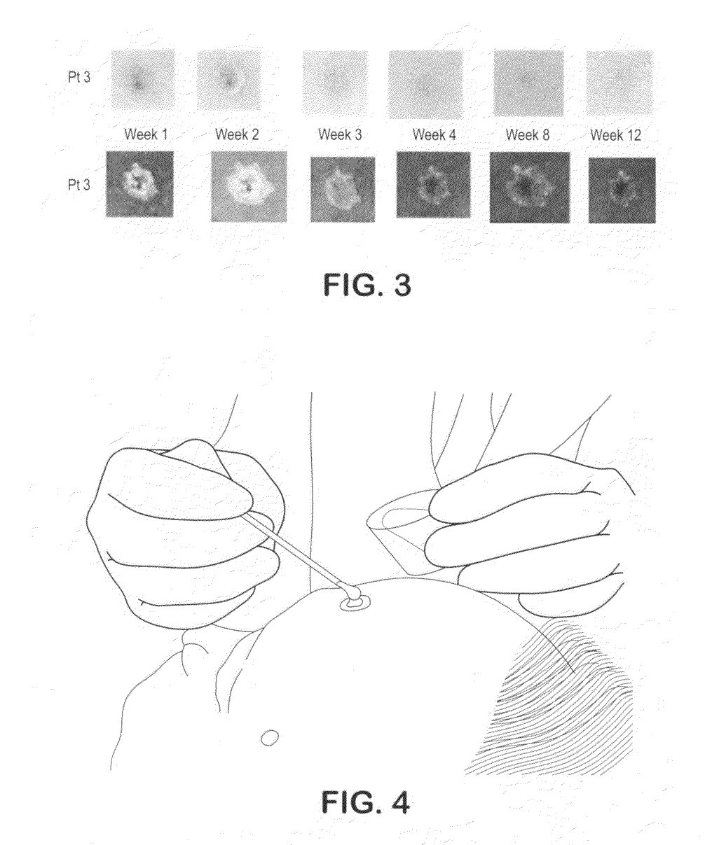

### DETAILED DESCRIPTION OF THE PREFERRED EMBODIMENTS

The preferred titanium dioxide-polymethylmethacrylate (PMMA) aqueous suspension according to the invention contains 2.5 wt % titanium dioxide and 97.5 wt % PMMA. This suspension is known for use in tattooing when it is desired that the tattoo be invisible in ordinary light but fluoresce under ultraviolet light. It is not known to use this suspension in the medical field as a skin marker for locating areas of interest. The PMMA is known to extend the fluorescing life of the titanium dioxide within the skin. Application of the titanium dioxide-PMMA suspension can, for example, be accomplished using swabs, it can be applied using band-aids which cover the biopsy area, it can be injected by way of a syringe, or sprayed from canisters.

The titanium dioxide-PMMA aqueous suspension is desirably applied together with an effective amount of Vitamin E oil, an effective amount aqueous aluminum chloride solution (bleeding inhibitor), Lidocaine and/or epinephrine. Vitamin E oil is desirably applied to the site of the titanium dioxide-PMMA application on a daily basis after initial application to extend the fluorescing life of the titanium dioxide, e.g., to twelve weeks or more.

It should be noted that zinc oxide, titanium oxide, zinc sulfate, lead carbonate and barium sulfate can be used to replace some or all of the titanium dioxide in the inventive aqueous suspension.

It is to be appreciated that the methods of the present invention using a titanium dioxide-PMMA aqueous suspension may be utilized in combination with one or more different products for use in marking the locations of initial biopsies. The following is a non-exclusive list of different exemplary techniques for marking the location of an initial biopsy, it being appreciated that these techniques may be adapted for use in any medical biopsy process.

1. Mixtures of the present invention may be combined with a local anesthetic, and injected as a combination when “numbing up” a patient prior to biopsy in order to mark the location of the biopsy. By way of example, and without limitation, the titanium dioxide-PMMA aqueous suspension may be combined with lidocaine and/or epinephrine, or with one or more other injectable anesthetics used in dermatology such as either the ester (benzocaine, procaine, novocaine) or amide (bupivicaine, prilocalne) classes, diphenhydramine, sodium chloride, or the like, in order to mark the location of the biopsy.

2. Mixtures of the present invention may be combined with aluminum chloride solution or other vehicle to be applied directly to the wound bed after the biopsy in order to mark the location of the biopsy.

3. Mixtures of the present invention may be provided in an adhesive band-aid or any other bandage, dressing, film, or strip(s) to be placed over the wound bed when the biopsy is complete in order to mark the location of the biopsy.

4. Mixtures of the present invention may be combined with saline (e.g., 0.9% sodium chloride), to be injected into the wound bed in order to mark the location of the biopsy.

5. Mixtures of the present invention may be combined with any inert/antibiotic/petrolatum/other gel, ointment, lotion, solution, foam, cream, paste and/or other vehicle to be applied to the biopsy wound in order to mark the location of the biopsy.

6. Mixtures of the present invention may be provided on any applicator device (e.g., a cotton-tip applicator used to apply (Drysol), such as a stick, brush, cotton-tip, wood, plastic, electrocautery tips, etc., and applied to the wound in order to mark the location of the biopsy.

7. Mixtures of the present invention may be combined with silver nitrate (used to stop bleeding), as a solution or an applicator/stick in order to mark the location of the biopsy.

8. Mixtures of the present invention may be combined with a ferric subsulfate solution (e.g., “Monsel's solution”) used to stop bleeding in order to mark the location of the biopsy.

9. Mixtures of the present invention may be combined into an alginate or other absorbent or hemostatic type dressing (Gelfoam, Surgicell), in order to mark the location of the biopsy.

10. Mixtures of the present invention may be combined into a needle to be stuck into the biopsy wound bed during injection with or without anesthetic, done during or after the biopsy in order to mark the location of the biopsy.

11. Mixtures of the present invention may be combined into a marker/pen/pencil device to mark the wound bed before or after biopsy.

12. Mixtures of the present invention may be combined into an aerosol product to be sprayed into the wound in order to mark the location of the biopsy.

It is to be understood that variations and modifications of the present invention may be made without departing from the scope thereof. It is also to be understood that the present invention is not to be limited by the specific embodiments, examples or applications disclosed herein. In particular, none of the methods or combinations of the present invention is limited to any particular field of human or animal medicine.

What is claimed is:

1. A marker composition for application to a human or animal for identification of a location of interest under ultraviolet light, the composition being invisible in ordinary light, the composition comprising an aqueous suspension of titanium dioxide and polymethylmethacrylate (PMMA).

2. The marker composition of claim 1, wherein the aqueous suspension comprises 2.5 wt % titanium dioxide and 97.5 wt % PMMA.

3. The marker composition of claim 2, including Vitamin E oil.

4. The marker composition of claim 2, including a aqueous aluminum chloride solution.

5. The marker composition of claim 2, including an effective amount of Lidocaine.

6. The marker composition of claim 2, including an effective amount of epinephrine.

7. The marker composition of claim 2, including an effective amount of an anesthetic.

8. A method of marking an external or internal location of a human being or animal for subsequent identification for medical purposes, said method comprising (a) applying a marker composition which comprises an aqueous suspension of titanium dioxide and polymethylmethacrylate at the location, and (b) irradiating the human or animal with ultraviolet light to cause fluorescing of said titanium dioxide.

9. The method of claim 8, wherein in step (a) the aqueous suspension is applied to an external location.

10. The method of claim 9, wherein in step (a) the aqueous suspension is injected beneath a surface of the location.

11. A method of marking a specific location on the skin of a person where a biopsy has been taken, said method comprising (a) applying a composition to the specific location which comprises an aqueous suspension of titanium dioxide, polymethylmethacrylate and Vitamin E oil, and (b) irradiating the person with ultraviolet light to cause fluorescing of the titanium dioxide at the specific location.

12. An applicator device for marking a location on a human or animal, said applicator device comprising an absorbent material impregnated with an aqueous suspension of titanium dioxide, polymethylmethacrylate and Vitamin E oil.

13. The applicator device of claim 12, comprising a band-aid with a pad impregnated with said aqueous suspension.

14. The applicator device of claim 12, comprising a swab having a cotton tip impregnated with said aqueous suspension.
